(19) United States
(12) Patent Application Publication
(10) Pub. No.: US 2009/0176740 A1
(43) Pub. Date: Jul. 9, 2009

(54) Treatment of neurological conditions by the co-administration of aniracetam and l-alpha glycerylphosphorylcholine

(76) Inventor: Dauglas James Phillips, II, Jupiter, FL (US)

Correspondence Address: Douglas J. Phillips II, 1100 Sioux Street, Jupiter, FL, 33458, US

(21) Appl. No.: 12/315,255

(22) Filed: Dec. 1, 2008

Related U.S. Application Data
(60) Provisional application No. 60/991,278, filed on Nov. 30, 2007.

(51) Int. Cl. A61K 31/66

(52) U.S. Cl. 514/75

(57) ABSTRACT

Aniracetam (1-[(4-methoxybenzoyl)]-2-pyrrolidinone) is co-administered with the acetylcholine precursor 1-alpha glycerylphosphorylcholine (Alpha GPC, choline alfoscerate, choline alphoscerate) to potentiate cognition enhancing effects in healthy subjects and patients suffering from neurological conditions including Alzheimer's Disease (AD), attention deficit disorder (ADD), Parkinson's Disease, schizophrenia, vascular dementia, post stroke aphasia, anxiety disorders, cerebral atrophy, chronic alcoholism, Down syndrome, dyslexia, and various other neurodegenerative conditions. The co-administration of aniracetam (and other racetam derivatives including oxiracetam) with the acetylcholine precursor 1-alpha glycerylphosphorylcholine decreases negative side-effects such as severe headaches while increasing the synthesis and release of the neurotransmitters acetylcholine and glutamate to facilitate proper brain functioning.

This application claims priority to my earlier filed provisional application Ser. No. 60/991,278 filed on Nov. 30, 2007.

### BACKGROUND OF THE INVENTION

Aniracetam falls into a category of neurological agents called ‘racetams’ that are analogs of piracetam. Piracetam was first discovered and synthesized by a team of researchers led by Dr Corneliu E. Giurgea in 1964 and has been used extensively throughout the world as a cognitive enhancer and to treat neurological conditions such as Alzheimer's Disease and senile dementia Since the original discovery of piracetam, analogs such as aniracetam and oxiracetam have been synthesized that are significantly more potent than the original piracetam. Aniracetam is one such racetam analog that is claimed to be four to ten times stronger than piracetam.

Aniracetam is often considered a member of the ampakine class of neurological compounds that interact with the glutamatergic AMPA receptor of the brain to increase memory functions, facilitate learning activities, and help modulate neurological conditions. Such neurobiological activity increases the release of the neurotransmitter glutamate that assists with neurological functions critical to normal and healthy brain operations.

L-alpha glycerylphosporylcholine (Alpha GPC, choline alfoscerate, choline alphoscerate, glycerylphosporylcholine) is an acetylcholine precursor derived from soy lecithin used to enhance memory and treat neurological disorders associated with neurodegeneration. L-alpha glycerylphosporylcholine readily crosses the blood brain barrier (BBB) and is considered one of the most efficacious of all the acetylcholine precursors in synthesizing the neurotransmitter acetylcholine. L-alpha glycerylphosporylcholine is the most bioavailable form of choline currently known. Further, L-alpha glycerylphosporylcholine has also been shown to be involved with the secretion of human growth hormone in the hypothalamus region of the brain where memory functions take place. Several neurological conditions, including Alzheimer's Disease, have been correlated with decreased levels of acetylcholine and hypothalamus degeneration.

### SUMMARY OF THE INVENTION

This invention is based on the discovery that members of the racetam family (including piracetam, aniracetam, and oxiracetam) lead to undesirable side effects when taken without an acetylcholine precursor such as 1-alpha glycerylphosphorylcholine (Alpha GPC). Such side effects include severe headaches and pain associated with acetylcholine depletion. When aniracetam is co-administered with 1-alpha glycerylphosphorylcholine, headaches and cerebral pain can be significantly avoided while maintaining neurological efficacy. The combination of derivatives of piracetam (including aniracetam and oxiracetam) with the acetylcholine precursor 1-alpha glycerylphosphorylcholine represents a novel class of chemical compounds with wide-ranging neurological benefits and minimal side-effects.

Aniracetam and 1-alpha glycerylphosphocholine work synergistically with one another to help counter the deleterious effects of acetylcholine depletion and neurodegeration often associated with age-related neurological conditions. The combination of aniracetam and 1-alpha glycerylphosphocholine is more efficacious in the treatment of neurological disorders when taken together than if either were administered alone.

Aniracetam and 1-alpha glycerylphosphocholine may be co-administered orally such as in capsule, tablet, powdered, or liquid form. A ratio of aniracetam to 1-alpha glycerylphospocholine appropriate and efficacious for cognitive enhancement and neurological treatment is 4:3. A capsule containing 400 mg of aniracetam and 300 mg of 1-alpha glycerylphosphorylcholine administered orally is one such form and method of co-administration.

What is claimed is:

1. The reduction of undesirable side effects such as headaches associated with administration of aniracetam can be alleviated in human subjects by the co-administration with 1-alpha glycerylphosphorylcholine. Co-administration of aniracetam and 1-alpha glycerylphosphorylcholine restores proper acetylcholine and glutamate neurotransmitter levels for proper brain functioning.

2. The process of treating neurological conditions including Alzheimer's Disease (AD), attention deficit disorder (ADD), memory impairment, Parkinson's Disease, schizophrenia, vascular dementia, post stroke aphasia, anxiety disorders, cerebral atrophy, chronic alcoholism, Down syndrome, dyslexia, and other neurodegenerative conditions using the combination of aniracetam and 1-alpha glycerylphosphorylcholine.

3. The process of enhancing or improving memory and brain functions associated with the acetylcholine neurotransmitter system in healthy human adults by the co-administration of aniracetam with 1-alpha glycerylphosphorylcholine.

4. The method of claim 1 wherein the mode of administration is oral.

5. The process of claim 2 wherein the mode of administration is oral.

6. The process of claim 3 wherein the mode of administration is oral.

7. The method of claim 1 wherein the mode of administration is intravenous.

8. The process of claim 2 wherein the mode of administration is intravenous.

9. The process of claim 3 wherein the mode of administration is intravenous.

10. The method of claim 1 wherein a racetam analog similar to aniracetam such as oxiracetam, pramiracetam, phenylpiracetam, etiracetam, levetiracetam, nefiracetam, rolziracetam, nebracetam, fasoracetam, coluracetam, brivaracetam, or seletracetam is combined with an acetylcholine precursor similar to 1-alpha glycerylphosphorylcholine including DMAE, choline bitartrate, choline citrate, cytidine diphosphate choline, centrophenoxine, or lecithin.

11. The process of claim 2 wherein a racetam analog similar to aniracetam such as oxiracetam, pramiracetam, phenylpiracetam, etiracetam, levetiracetam, nefiracetam, rolziracetam, nebracetam, fasoracetam, coluracetam, brivaracetam, or seletracetam is combined with an acetylcholine precursor similar to 1-alpha glycerylphosphorylcholine including DMAE, choline bitartrate, choline citrate, cytidine diphosphate choline, centrophenoxine, or lecithin.

12. The process of claim 3 wherein a racetam analog similar to aniracetam such as oxiracetam, pramiracetam, phenylpiracetam, etiracetam, levetiracetam, nefiracetam, rolziracetam, nebracetam, fasoracetam, coluracetam, brivaracetam, or seletracetam is combined with an acetylcholine precursor similar to 1-alpha glycerylphosphorylcholine including DMAE, choline bitartrate, choline citrate, cytidine diphosphate choline, centrophenoxine, or lecithin.
